(19) United States
(12) Patent Application Publication
(10) Pub. No.: US 2015/0313542 A1
(43) Pub. Date: Nov. 5, 2015

(54) WEARABLE ELECTRONICS

(71) Applicant: Neumitra Inc., Boston, MA (US)

(72) Inventors: Robert F. Goldberg, Boston, MA (US); Shailendra Yadav, Lexington, MA (US); Safiyy Momen, Medford, MA (US)

(73) Assignee: Neumitra Inc., Boston, MA (US)

(21) Appl. No.: 14/702,208

(22) Filed: May 1, 2015

Related U.S. Application Data
(60) Provisional application No. 61/987,346, filed on May 1, 2014.

(51) Int. Cl. A61B 5/00; A61B 5/0205; A61B 5/04; A61B 5/08; A61B 5/021; G04B 47/06; A61B 5/024

(52) U.S. Cl. CPC A61B 5/681; G04B 47/063; A61B 5/0205; A61B 5/02405; A61B 5/024; A61B 5/0816; A61B 5/02055; A61B 5/021; A61B 5/04004

(57) ABSTRACT

A wearable article includes a flexible band comprising one or more band segments; one or more biosensors located in the flexible band; one or more processing units located in the flexible band; and at least one connecting mechanism configured to connect at least one end of the flexible band to a housing of a watch face. A method of monitoring a physiological state of a wearer via a flexible band connected to a watch face housing worn at the wearer's wrist includes: receiving, at one or more processing units located in the flexible band, sensor data from one or more biosensors located in the flexible band; and analyzing the received sensor data, via the one or more processing units, to compute a score representative of a physiological state of the wearer.

### CROSS-REFERENCE TO RELATED APPLICATIONS

This application claims the benefit under 35 U.S.C. §119(e) of U.S. Provisional Patent Application No. 61/987,346, filed May 1, 2014, and entitled “WEARABLE ELECTRONICS DESIGNED TO FIT VARIOUS ANATOMIES WITH RECOMBINED COMPONENTS,” which is hereby incorporated by reference in its entirety.

### BACKGROUND

Biosensors measure physiological signals representative of a person's emotional state. This information may be used as a type of biofeedback, which may aid a person to be aware of and alter their response to stressful situations or to avoid those situations. This information may also be used for diagnosis, detection, monitoring or treatment of physiological disorders.

Biosensors may measure physiological signals such as temperature, pulse rate or sweat production of a user. The biosensors may be worn by a user such that they can measure those signals over time as the user participates in various activities. Such measurements produce data that may be analyzed to determine a user's biological and/or health state, such as if the user has a higher than average temperature.

### SUMMARY

One type of embodiment is directed to a wearable article comprising: a flexible band comprising one or more band segments; one or more biosensors located in the flexible band; one or more processing units located in the flexible band; and at least one connecting mechanism configured to connect at least one end of the flexible band to a housing of a watch face.

Another type of embodiment is directed to a method of monitoring a physiological state of a wearer via a flexible band connected to a watch face housing worn at the wearer's wrist, the method comprising: receiving, at one or more processing units located in the flexible band, sensor data from one or more biosensors located in the flexible band; and analyzing the received sensor data, via the one or more processing units, to compute a score representative of a physiological state of the wearer.

Another type of embodiment is directed to a watch band comprising: a band of stretchable material; a first electronic component and a second electronic component disposed within the band, the first electronic component comprising a biosensor; and a stretchable electronic interconnect between the first electronic component and the second electronic component.

Another type of embodiment is directed to a wearable article comprising: a flexible band; one or more biosensors located in the flexible band; one or more processing units located in the flexible band; and at least one attachment mechanism configured to attach the flexible band to a wrist watch.

Another type of embodiment is directed to a wearable article comprising: a detachable module configured to attach to and to allow detachment from a housing of a watch face; one or more biosensors located in the detachable module; and one or more processing units located in the detachable module.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates an exemplary embodiment of a flexible band with embedded electronic components;

FIG. 2 illustrates an exemplary embodiment of a wearable article including the flexible band of FIG. 1 connected to a watch face housing;

FIG. 3 illustrates a different view of the exemplary wearable article of FIG. 2;

FIG. 4 illustrates a different configuration of the exemplary wearable article of FIGS. 2 and 3;

FIGS. 5A and 5B illustrate a configuration of the exemplary wearable article of FIG. 4 worn by a wearer;

FIGS. 6A-6F illustrate an exemplary embodiment of an assembly of electronic components that may be included in a wearable article;

FIG. 7 illustrates exemplary design criteria for some embodiments;

FIG. 8 illustrates exemplary contoured band embodiments;

FIGS. 9A-9C illustrate exemplary biosensor module embodiments; and

FIG. 10 illustrates an exemplary bioband embodiment.

### DETAILED DESCRIPTION

Some embodiments relate to designs and techniques for incorporating biosensor devices into a wearable article for making physiological measurements while being worn by a user (e.g., a person). These designs and techniques may provide user comfort, promoting use of the biosensors in many settings, including during normal work and personal activities. Alternatively or additionally, the wearable articles may hold the biosensors in a way that promotes accuracy of the measurements by the bio sensors. By making accurate biosensor data collected during numerous activities of a user available for computerized processing, computerized tasks may be adapted based on a current biological state, such as stress level, of the user.

Some embodiments relate to a wearable article that houses one or more electronic components such as biosensors. Some embodiments relate to a wearable article housing one or more electronic components that maintains one or more of the electronic components in contact with the wearer's wrist. In some embodiments, the wearable article may include a transmitter for communicating a current state of the wearer to a portable electronic device (e.g., a smart phone, tablet, PDA, etc.) or other computing device that may be programmed to perform different operations depending on the state of a user. Alternatively or additionally, the wearable article may include components, such as a processor and/or memory, that may process physiological measurements and determine a state of the wearer. The processor may update information stored in the memory that is used in mapping physiological information to a current state.

In some embodiments, the wearable article may be a wrist-worn watch, or may be configured to attach to a wrist-worn watch or to a part of a wrist-worn watch. In some embodiments, the wearable article may be a band, with or without connection to a watch. In some embodiments related to a wrist-worn watch, some or all of the electronic components related to the biosensor device(s) (e.g., sensor(s), one or more batteries, transmitter(s), processing unit(s), etc.) may be distributed in the wrist band as opposed to being housed in the watch face. In some embodiments, to accommodate electronic components located where the band crosses the underside of the wrist, the clasp for attaching the band to the wrist may be located offset from that location, in any other suitable location such as in either or both if the sides of the band, and/or at the junction between the band and the watch face.

In some embodiments, biosensor device(s) incorporated into a wearable article may include components such as those described in U.S. patent application Ser. No. 13/040,816, filed Mar. 4, 2011, and entitled “Devices and Methods for Treating Psychological Disorders,” and/or those described in U.S. Provisional Patent Application Ser. No. 61/310,280, filed Mar. 4, 2010, and entitled “A device for monitoring and treating mood disorders.” The disclosures of both of these applications are incorporated herein by reference in their entireties. For example, biosensor device components may include one or more: (micro)processors, memories and/or other data storage devices, transmitters, receivers, power sources, displays, motion sensors (e.g., accelerometers), global positioning systems, clocks, and/or sensors, such as heart rate, pulse rate, beat-to-beat heart rate variability, electrocardiography, respiration rate, skin temperature, core body temperature, heat flow off the body, electrodermal activity (galvanic skin response), electromyography, electroencephalography, electrooculography, blood pressure, hydration level, muscle pressure, activity level, body position, optical reflectance of blood vessels, oxygen saturation sensors, etc.

In some embodiments, physiological data obtained by a biosensor device may be analyzed and processed by a local processor of the biosensor device to determine the wearer's health, including, e.g., the wearer's physical, mental, and/or emotional state. In some embodiments, the wearer may use this information to track their health state over time as the biosensor device acquires additional physiological data. Analysis and processing of physiological data in some embodiments may include determining a health state of a wearer based on the physiological data. The health state may include one or more parameters that indicate an aspect of the wearer's health, such as the wearer's current stress level, for example. Any suitable computation technique may be used to compute a health state value from a physiological signal. Exemplary techniques include those described in U.S. patent application Ser. No. 13/040,816 and/or in U.S. Provisional Patent Application Ser. No. 61/310,280, incorporated herein by reference.

In some embodiments, a processor attached to or embedded in the wearable article may receive physiological measurements obtained by the biosensor device and process the measurements to generate health state information. Such processing in some embodiments may include correlating physiological data to a health state based on previously acquired physiological data. The previously acquired data may correspond to known health state information, and in some embodiments current physiological data may be compared and/or mapped to the previously acquired data to determine current health state information.

In some embodiments, the health state may correspond to a particular health value. The health value may have any suitable form and, in some embodiments, may represent a value of a single health-related characteristic. The value assigned may indicate the degree to which data associated with the wearer indicates that characteristic is present. For example, specific physiological data may correlate to a range of stress levels, and a stress level of the wearer corresponding to current physiological data may be identified by mapping the current physiological data to the specific physiological data. The current physiological data may be similar to the specific physiological data corresponding to the identified stress level. A health state value may indicate a stress level or a degree to which the wearer is stressed, for example. In some embodiments, the health state value may indicate multiple characteristics, such as, e.g., stress and activity level. By acquiring physiological data over time, in some embodiments the wearer's stress status along a range of stress levels may be tracked over time and may include a high stress level and a low stress level, such as may be characteristic of a calm and/or relaxed state.

The wearer may use such stress level information in any suitable way, such as to make decisions that may impact their overall well-being. To support such uses, in some embodiments the wearable article may include an output mechanism, such as a haptic device. Alternatively or additionally, the wearable article may include a transmitter and/or receiver for communication with a smart phone or other portable computing device that may process data and/or use information generated within the wearable article. In such embodiments, one or more of the biosensors described above and the analysis of the physiological data to determine a health state may be part of a biofeedback process.

In some embodiments, information regarding health states of a user determined based at least in part on sensor data from a wearable article such as embodiments described herein may be input to any suitable software application executing on a computing device, which may include one or more software applications having functions other than processing physiological data and providing biofeedback to a user. For example, in some embodiments, health state information may be provided to any type of software application (examples include e-mail applications, web browsers, office tool applications, gaming applications, operating systems, and/or any other suitable application) to make the application aware and responsive to the health state of the user. Exemplary enhanced functionality of an application that is health-state-aware may include adapting the visual display of the application, such as colors, themes, etc., based on the user's health state, controlling operations executed by the application, such as playing music, scheduling events, blocking/allowing phone calls, and/or otherwise controlling execution of tasks that may impact and/or be impacted by the user's health state, and/or any other suitable health-state-aware functionality. Further examples of uses that may be made of health-state information provided by a biosensor device such as embodiments described herein are provided in U.S. Provisional Patent Application Ser. No. 62/002,758, filed May 23, 2014, and entitled “OPERATING SYSTEM WITH COLOR-BASED HEALTH STATE THEMES.” The disclosure of that application is hereby incorporated by reference herein in its entirety.

In some embodiments, a local data file on the biosensor device, or a data file stored in another location, may store the previously acquired physiological data as a profile. The profile may be derived from physiological data corresponding to one or more individuals. The profile may be updated to reflect additional physiological data and health state information resulting from analysis of the additional physiological data. In this manner, the profile in some embodiments may reflect changes in how physiological data corresponds to health state information and may improve identification of a health state as additional physiological data is included in the profile. In some embodiments, the profile may reflect physiological data for the wearer, and identifying a health state based on current physiological data of the wearer may include correlating the current physiological data to the previously acquired physiological data stored in the wearer's profile. As additional physiological data is acquired and processed to determine a current health state, updates to the profile may include at least a portion of the physiological data and the identified health state. In this manner, in some embodiments the profile may become specific to the wearer as additional physiological data for the wearer is acquired and health state information is identified.

In some embodiments, the profile may reflect physiological data acquired from a population of individuals and may include statistical information for physiological data associated with health state information of the population. Such statistical information may include a range, averages, and/or standard deviations of physiological data and health state values. Determining a health state for the wearer in some embodiments may include comparing current physiological data to the health statistical information and health information stored in the profile. The statistical information may provide physiological data statistics for a population corresponding to identified health states, and in some embodiments a wearer's health state may be identified by comparing current physiological data to the physiological data statistics. Updates to the profile may reflect changes to the statistical information and health information of the population as physiological data for individuals among the population is acquired. Additionally or alternatively, a profile based on population physiological data may be updated with physiological data and identified health state information of the wearer. In this manner, the profile may begin as a general or default profile and gradually adapt to include data specific to the wearer as physiological data is obtained by the biosensor device.

In some embodiments, the profile may also include contextual information associated with physiological data measured. The contextual information may include time, location, and/or activity that an individual is performing associated with the physiological data. The contextual information may be passed by a component of the biosensor device and/or received from another device such as a smartphone or other portable electronic device. The processor of the biosensor device may process context information to determine a current context for the user, and determining a health state may include analyzing the current context information associated with physiological data obtained by the biosensor device. The profile information may include the contextual information and related physiological data such that physiological data corresponding to a specific context can be retrieved and compared to current physiological data. A current context of the wearer may be used to select a subset of previously acquired physiological data and associated health state information.

In some embodiments, determining a current health state of the wearer may include comparing current physiological data to the subset of physiological data representing a similar user context. In this manner, a current health state of the wearer may be represented as a comparison to other occurrences when the wearer was in a similar situation. For example, context information may indicate that a wearer is commuting to work based on time and/or geographical information. A subset of physiological data stored in the profile may be selected by identifying physiological data from the profile associated with contextual information indicating similar time and/or geographical information, and a health state may be identified based on the subset of physiological data and indicate a relative health state in comparison to other times the wearer was on his morning commute. In some embodiments, patterns associated with a wearer's health under certain contexts may be identified by analyzing physiological data associated with a particular context. For example, such an analysis may indicate a pattern of an individual becoming more stressed while commuting.

It should be appreciated that the foregoing description is by way of example only, and some embodiments are not limited to providing any or all of the above-described functionality, although some embodiments may provide some or all of the functionality described herein.

Features described herein can be implemented in any of numerous ways, and are not limited to any particular implementation techniques. Thus, while examples of specific implementation techniques are described below, it should be appreciated that the examples are provided merely for purposes of illustration, and that other implementations are possible.

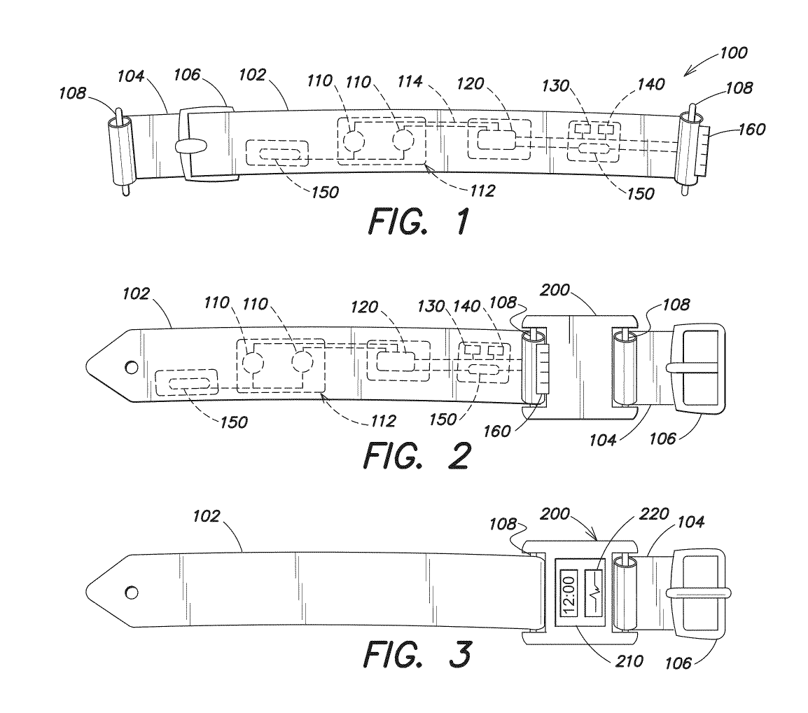

Illustrated in FIG. 1 is an exemplary embodiment of a wearable article designed to attach to a watch face housing, e.g., for a wrist watch. The exemplary wearable article of FIG. 1 includes a flexible band 100, which is formed from separate band segments 102 and 104 joined by clasp 106. This is merely an example; other embodiments may have different numbers of band segments forming band 100, and in some embodiments band 100 may be formed of only one band segment without a clasp. In some embodiments, as illustrated in FIG. 1, one or more of the ends of band 100 may have a connecting mechanism, such as pins 108 or loops, hooks or other structures that can be attached to such pins, configured to connect the end of the band to a housing of a watch face (not shown in FIG. 1). Pins 108 may be spring pins as in a conventional watch face housing, or may have any other suitable configuration. In other embodiments, the wearable article including flexible band 100 and any of the embedded components described below may not be configured to connect to a watch face housing, but may instead by configured to connect to a different device, or may not be configured to connect to any other device, as in the case of a band for wearing on its own.

Band 100 may be made of any suitable material such that band 100 is flexible, e.g., for wrapping around a wearer's wrist, or other body part. In some embodiments, band 100 may be made of leather; in other embodiments, band 100 may be made of fabric, rubber, flexible plastic, metal and/or plastic links, and/or any other suitable material or combination of materials. Clasp 106 may be any suitable form of band clasp, and connecting mechanism 108 may be any suitable form of mechanism for connecting band 100 to a watch face housing, including any suitable known or later developed form. The exemplary forms of clasp 106 and connecting mechanism 108 shown in FIG. 1 are provided merely for purposes of illustration, and are not intended to be limiting.

In some embodiments, one or more biosensors 110 may be located in band 100, and each biosensor may be attached to, protruding from, or embedded beneath one or more surfaces of band 100. For example, in some embodiments as illustrated in FIG. 1, biosensor(s) 110 may be fixed on a substrate 112, such as a flat piece of plastic, a printed circuit board (PCB), or other suitable substrate, which may be embedded in band 100 between two strips of leather or other material forming the outer surfaces of band 100. Depending on the type of biosensor(s) being included and a tradeoff between placing the biosensor(s) in direct contact with the skin of the wearer vs. concealing the biosensor(s) from view and environmental contamination when band 100 is not being worn, in some embodiments biosensor(s) 110 may be covered by the surface material of band 100, while in other embodiments, one or more openings may be formed in the surface material through which biosensor(s) 110 may protrude, or biosensor(s) 110 may be attached to the outside surface of band 100.

In some embodiments, substrate 112 may form an “island” within band 100, and other electronic components may form or occupy other “islands” separated from substrate island 112 within band 100. For example, in some embodiments, one or more processing units 120 such as one or more microprocessors may be located in band 100, on the same substrate 112 and/or on one or more separate substrate islands from biosensor(s) 110. In some embodiments, biosensor(s) 110 may have one or more electronic connections 114 to carry sensor data from biosensor(s) 119 to processing unit(s) 120, and/or to carry command data from processing unit(s) 120 to biosensor(s) 110. In some embodiments, electronic connections 114 between electronic components located in band 100 may be formed of any suitable flexible and/or stretchable material(s), to accommodate movement of the wrist and/or flexing and/or stretching of band 100 while maintaining the electronic connections between components for continuous power and/or data flow. In some embodiments, rigid islands such as PCB islands supporting electronic components may be separated by flexible and/or stretchable connecting regions made of any suitable elastomer (e.g., rubber), through which electrical connections may be flexibly routed. For example, in some embodiments, conductive electrical connections such as copper lines may traverse the flexible connecting regions in folded, coiled, and/or spiraling configurations, and/or in any other suitable configuration allowing the conductive lines to flex and/or stretch along with the surrounding material in the regions connecting the rigid islands, without breaking or creating discontinuities in power and/or data transmission between islands.

In some embodiments, processing unit(s) 120 may also have one or more associated storage media, which may be any suitable form of processor-readable storage media, located in band 100, either in proximity to processing unit(s) 120, such as on the same integrated circuit and/or on the same substrate island within band 100, or in a different location in band 100 with any suitable connection(s) to processing unit(s) 120. In some embodiments, the storage media may store processor-readable instructions executed by the processing unit(s) 120 to control biosensor(s) 110, to receive and process sensor data from biosensor(s) 110, and/or to perform any other suitable function(s), other examples of which are described herein. In some embodiments, embedded storage media may also be used for volatile and/or non-volatile storage of data such as sensor data and/or other data about a wearer.

In some embodiments, processing unit(s) 120 may execute stored instructions to receive sensor data from biosensor(s) 110 and to analyze the received sensor data to identify a physiological and/or psychological state of the wearer. This may be done in any suitable way using any suitable technique(s). For example, in some embodiments, the analysis may be performed using any of the techniques and any of the sensor data described in U.S. patent application Ser. No. 13/040,816 and/or in U.S. Provisional Patent Application Ser. No. 61/310,280, incorporated herein by reference. Other exemplary techniques are described above. In some embodiments, processing unit(s) 120 may determine a level of stress exhibited by the wearer, based on the sensor data collected by biosensor(s) 110. In some embodiments, this stress level may be represented by processing unit(s) 120 as a numerical score or a category. Alternatively or additionally, in some embodiments raw sensor data may be transmitted for processing remotely from band 100, and/or some front-end processing may be performed in band 100 while further analysis may be performed remotely.

In some embodiments, band 100 may include one or more wireless transmitters 130, such as a radio frequency (RF) transmitter, for transmitting data from processing unit(s) 120 and/or biosensor(s) 110 to be analyzed and/or otherwise processed remotely from band 100. In some embodiments, this may include transmission of data indicating the physiological and/or psychological state of the wearer identified by analyzing the biosensor data, such as the stress level determined by processing the sensor data via processing unit(s) 120. Alternatively or additionally, raw sensor data may be transmitted in some embodiments. The data may be transmitted to any suitable receiving device for any suitable further use of the data. For example, in some embodiments, sensor data and/or processed data such as scores may be transmitted to a separate device carried by the wearer, such as a laptop, a tablet, a smart phone, a PDA, etc. In other embodiments, data may be transmitted to a device located elsewhere, such as a desktop computer or other device via a local wireless or Internet or cellular data connection. Alternatively or additionally, in some embodiments transmitter 130 may be used to send raw and/or processed data to a suitable receiving device in a watch face housing or other device connected to band 100. In some embodiments, the transmitted data may be used to supply alerts to the wearer and/or to another recipient, related to the wearer's physiological and/or psychological state, e.g., as described in U.S. patent application Ser. No. 13/040,816 and/or in U.S. Provisional Patent Application Ser. No. 61/310,280, incorporated herein by reference.

In some embodiments, band 100 may include a vibration-generating device 140, controlled by processing unit(s) 120. Device 140 may be any suitable form of device capable of delivering a vibration stimulus to the wearer of band 100, including any suitable known or later developed form. Although exemplary FIG. 1 depicts vibration-generating device 140 as being housed on the same island as transmitter 130 within band 100, this is merely an example and is not required. In general, any component described herein may be located on any substrate island, separately or in combination with any other component, as embodiments are not limited in this respect. Moreover, in some embodiments, the islands may be formed without a separate or rigid substrate. For example, in some embodiments, one or more electronic components forming one or more islands may be attached directly to a flexible circuit.

In some embodiments, processing unit(s) 120 may activate vibration-generating device 140 to deliver vibration stimuli to alert the wearer to the wearer's physiological and/or psychological state as identified based on the data from biosensor(s) 110. For example, in some embodiments, a vibration alert may be delivered to the wearer when the wearer's stress level is determined to exceed any suitably designated threshold, which may be specified by the user or as a default standard.

In some embodiments, band 100 may further include one or more batteries 150 for providing power to processing unit(s) 120, to biosensor(s) 110, and/or to any other suitable electronic component(s) embedded in band 100, via one or more electrical connections carrying power from the battery to the electronic component(s). Batteries 150 may be located in any suitable position within band 100, on isolated and/or shared islands with respect to other components, and in some embodiments may be in distributed locations to balance weight and/or bulk across band 100. Alternatively or additionally to powering electronic components embedded in band 100, in some embodiments one or more batteries 150 in band 100 may provide power to the watch face housing when connected via connecting mechanism 108, via one or more suitable electrical connections. In some embodiments, alternatively or additionally to wireless transmitter 130, band 100 may include one or more electronic interfaces 160 configured to deliver data and/or battery power to the watch face housing. Electronic interface 160 may be of any suitable form, including any suitable known or later developed form of interface for data and/or power connection.

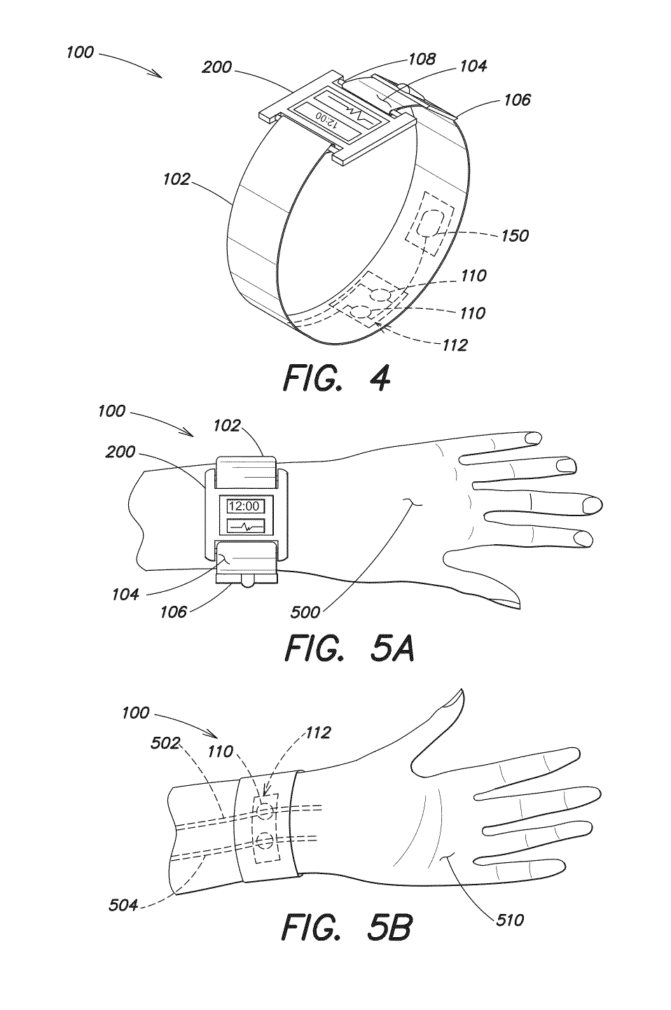

FIG. 2 illustrates a configuration of the exemplary wearable article from FIG. 1 in which the band 100 is connected via pins 108 to an exemplary housing 200 of a watch face, and band segments 102 and 104 have been separated from each other by unfastening the clasp 106. In this example, electronic interface 160 connects to watch face housing 200 via any suitable connection port, such that data and/or power from components within band 100 may be communicated to components in watch face housing 200.

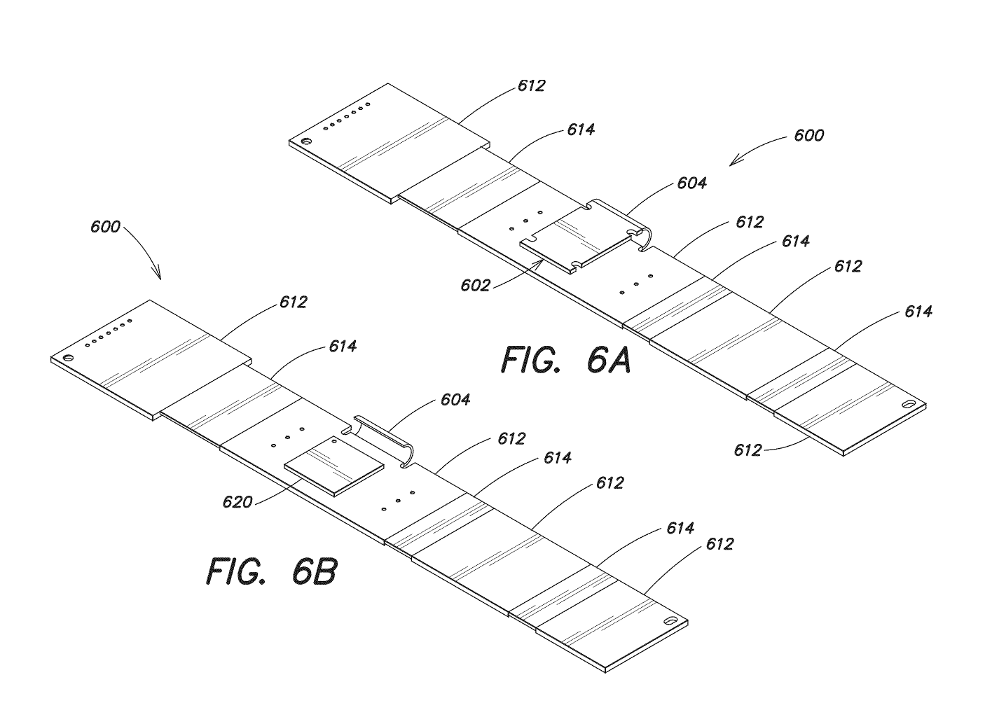

FIG. 3 illustrates the example wearable article from FIG. 2, now flipped over to the upper side on which the watch face display 210 in housing 200 is visible as it would be to the wearer when the article is strapped to the wearer's wrist. Exemplary display 210 includes a panel 220 in which physiological data and/or alerts based on biosensor data may be displayed to the wearer in some embodiments. Such data may be received at watch face housing 200 via interface 160 from band 100, and in some embodiments may be processed by processing unit(s) 120 within band 100 and/or by one or more other processing units within watch face housing 200.

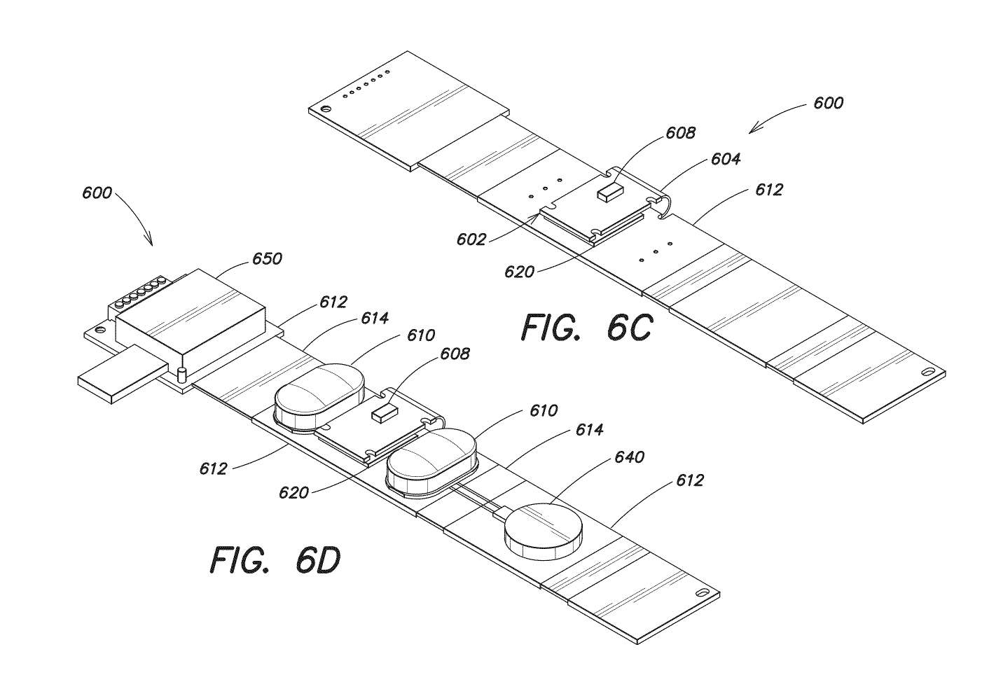

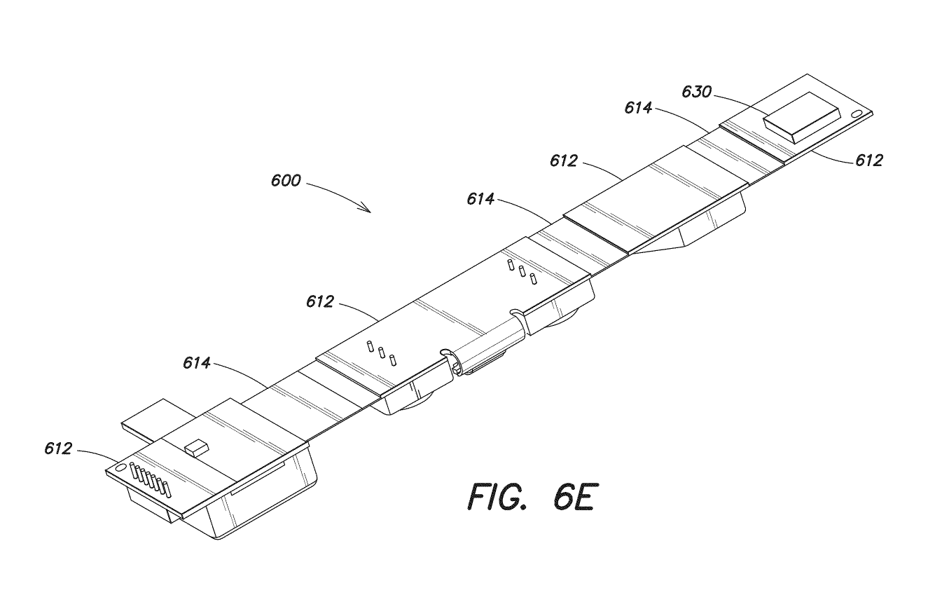

Illustrated in FIG. 4 is the example wearable article from the previous Figures, with the band 100 wrapped around in an annular configuration such that both ends of band 100 are connected to watch face housing 200 via pins 108 and clasp 106 fastens band segments 102 and 104 together. In some embodiments, one or more biosensors 110 may be located at a position in band 100 such that they are on the opposite side of the annular configuration from watch face housing 200, such that when watch face housing 200 is connected to band 100 and worn on the upper side of the wearer's wrist (i.e., by the back of the hand 500) as in FIG. 5A, the biosensor(s) 110 are positioned against the underside of the wearer's wrist (i.e., by the palm of the hand 510) as in FIG. 5B. In some embodiments, one or more biosensor(s) 110 may be located in band 100 at a position that contacts the wearer's wrist proximate the wearer's radial artery 502 and/or ulnar artery 504. In some embodiments, one or more sensors may be located proximate the radial artery 502 and one or more other sensors may be located proximate the ulnar artery 504. In some embodiments, one or more sensors may be located proximate the radial and/or ulnar artery, while one or more other sensors may be located at one or more other different positions in band 100. As also shown in FIG. 4 and FIG. 5A, in some embodiments, the clasp that separates band 100 into band segments may be located at a position offset from the underside of the wearer's wrist, e.g., making room for one or more biosensors at the underside of the wrist.

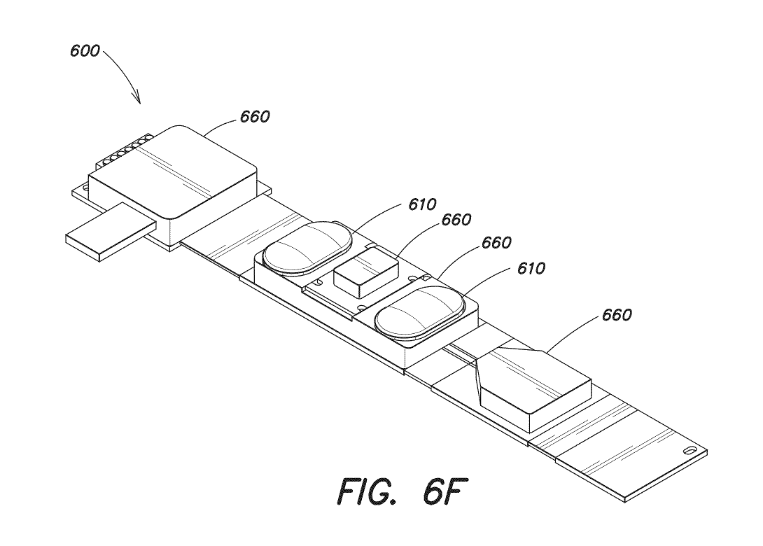

Illustrated in FIGS. 6A-6F is another exemplary embodiment of an assembly 600 of electronic components that may be embedded in a flexible band as described above. The views in FIGS. 6A-6F depict assembly 600 at various levels of deconstruction to illustrate an exemplary configuration of connected components. It should be appreciated, however, that this is merely one illustrative example. Some embodiments in accordance with the present disclosure are not limited to any particular configuration of components, and are not limited to inclusion of all or any particular set of components illustrated in the example of FIGS. 6A-6F.

As shown in FIG. 6A, exemplary assembly 600 includes a number of rigid islands 612 separated and connected in a linear configuration by flexible connecting regions 614. In addition, this exemplary assembly 600 includes a rigid island 602 connected to one of the islands 612 by a flexible connecting region 604 that curves to flip island 602 over its connected island 612. As this example illustrates, islands may be positioned and connected in any suitable configuration and are not limited to linear configurations, nor are they limited to inhabiting the same plane. Exemplary assembly 600 includes a microprocessor 620 attached to the island 612 below island 602, as illustrated in FIG. 6B (where island 602 is removed from the view for ease of viewing microprocessor 620). As shown in FIG. 6C, a heart rate sensor 608 occupies island 602 above microprocessor 620, thus allowing microprocessor 620 to occupy space on assembly 600 while still allowing a biosensor to be placed in the same linear position with respect to the wearable band and the wearer's wrist or other body part.

FIG. 6D illustrates the placement of additional electronic components on islands 612 of exemplary assembly 600, including a battery 650, two galvanic skin response (GSR) sensors 610, and a vibration-generating motor 640 for haptic feedback to the wearer. Again, it should be appreciated that the illustrative configuration in FIG. 6D is merely one example and is not intended to be limiting. Various embodiments may have different numbers and/or types of electronic components than depicted in FIG. 6D, and there may be any suitable number of each type of component in any suitable configuration. For example, the number of sensors 610 is not limited to two, and other embodiments may have 0, 1, 2, 3, or any other suitable number of sensors 610.

In the exemplary configuration of assembly 600, battery 650 and motor 640 occupy separate islands 612 from biosensors 608 and 610 and microprocessor 620; however, some embodiments are not limited in this respect. As described above, stretchable conductive connections in flexible connecting regions 614 may carry power, data, and/or commands between islands 612 and between the electronic components housed on them. In accordance with the placement of the biosensors 608 and 610, the view shown in FIG. 6D is of the surface of assembly 600 that will face the wearer's wrist or other body part when embedded in the wearable article such as a flexible band. Exemplary assembly 600 also includes a wireless transmitter (e.g., Bluetooth) device 630, which as illustrated in FIG. 6E is attached to the opposite surface of an island 612, on the side of assembly 600 that will face away from the wearer's wrist or other body part, e.g., for less obstructed data transmission. However, this is not required; neither is it required for other electronic components to be attached on the same side of such an assembly as each other, as the embodiment in FIGS. 6A-6F is merely an example.

FIG. 6F illustrates the full assembly 600, again viewing the side designed to face the wearer's wrist or other body part. Shown in this Figure are a number of caps 660, which may be formed of any suitable protective material (e.g., plastic, metal, etc.) for protecting the respective electronic components that they house. In the example assembly 600, GSR sensors 610 protrude through openings in the surrounding cap 660, which protects and isolates the underlying microprocessor 620, but allows GSR sensors 610 to protrude for more direct contact with the wearer's skin. Likewise, the surface (e.g., leather surface) of the band in which assembly 600 is embedded may include corresponding openings for GSR sensors 610 to contact the wearer's skin in some embodiments, or in other embodiments the band surface may cover sensors 610 to create an unbroken aesthetic to the wearable article. In some embodiments, one or more biosensor pads may have built-in compliance to maintain contact between the sensor(s) and the wearer's body surface as the body surface changes position, shape, etc. In some embodiments, one or more spring-like materials and/or mechanisms may be located under one or more sensors (i.e., on the opposite side of the sensor from the wearer's body surface) and may exert a force tending to press the sensor(s) toward and/or into the wearer's body surface. Alternatively or additionally, in some embodiments the flexible band housing the sensor(s) may be designed to be worn tight against the wearer's body surface and with suitable tension and elasticity to tend to maintain contact between the sensor(s) and the wearer's body surface as the body surface moves.

Some embodiments relate to a wearable article, incorporating electronic components, that is configured differently for wearers of different physical dimensions. In some embodiments, different configurations may be provided to align one or more of the electronic components with one or more corresponding anatomical structures in the wearer. In some embodiments, such anatomical structures may include one or more blood vessels, glands, and/or organs. In some embodiments, a wrist-worn article may be configured to align one or more sensors with the wearer's radial and/or ulnar articles, and/or eccrine sweat glands.

Some embodiments relate to methods of fitting one or more electronic components such as biometric sensors to a portion of a wearer's anatomy, e.g., by taking physical measurements of the wearer, and configuring a wearable article to house the components in a configuration that suitably aligns the components with the wearer's anatomical structures based on the wearer's physical measurements. For example, in some embodiments, a database or other relational structure such as one or more tables may be maintained that correlate different numerical measurements of a wearer's anatomical structures, such as a wearer's wrist circumference, to different particular sizes and/or shapes of a wearable article and/or to different particular configurations of electronic components housed within the wearable article.

In some embodiments, the biosensor device components may be arranged in the wearable article to promote accuracy of measurement by aligning sensors with anatomical structures from which they are configured to take measurements, and/or to promote wearability, e.g., by facilitating comfort, style, discreteness, etc. In one particular example, an article designed to be worn on the wrist may be configured to provide sensors held in place against the radial and/or ulnar arteries in the wrist. In some embodiments, a wearable biosensor article may be tailored differently to wearers of different physical dimensions and/or other constraints. In some embodiments, such tailoring may involve rearranging device components of the wearable article with respect to each other (e.g., rather than merely scaling the entire article by expanding or shrinking it while maintaining the relative positions of the various components), and/or may involve adding and/or removing device components and/or reshaping individual components to accommodate a particular size and/or fit of the article for a particular wearer. Rearranging, adding, removing, and reshaping components are referred to herein by the umbrella term “recombining.”

Some embodiments of designs for wearable technologies to monitor the physiology may depend on person-specific variables such as height, weight, and/or other physical dimensions and constraints. The wrist is an illustrative example. The nervous system, vasculature, sweat glands, skeletal system, and various receptors all converge near to the skin surface. But wrists come in a wide variety of sizes, shapes, hair distribution etc. Therefore some embodiments, in order to fit well, and/or for high data quality, may accommodate the various ways the anatomy and physiology are structured. In some embodiments, materials used to create the wearable biosensor article may be designed to fit, stretch, and/or conform while circuits may be configured to adapt to specific dimensions and functions.

In some embodiments, a wearer's wrist (or other suitable body location) may be measured to determine the best way to fit a wearable article to the wearer's anatomy with the sensor(s) and/or other electronic components of the article maintained in their desired positions in relation to the wearer's relevant anatomical structures. In some embodiments, a range of sizes and/or shapes/styles of the wearable article may be pre-fashioned for different wearer size categories. In other embodiments, the size, shape and configuration of the wearable article and/or its electronic components may be custom tailored to each individual wearer. In some embodiments, different electronic components may be selected for inclusion in different wearable articles for wearing on different parts of the body, and/or for different individuals for whom different types of measurements have different levels of applicability and/or importance. In some embodiments, the wearer may choose desired electronic components for inclusion in the wearable article, and/or may choose size and/or form constraints for the wearable article, into which suitable electronic components may then be fit adaptively.

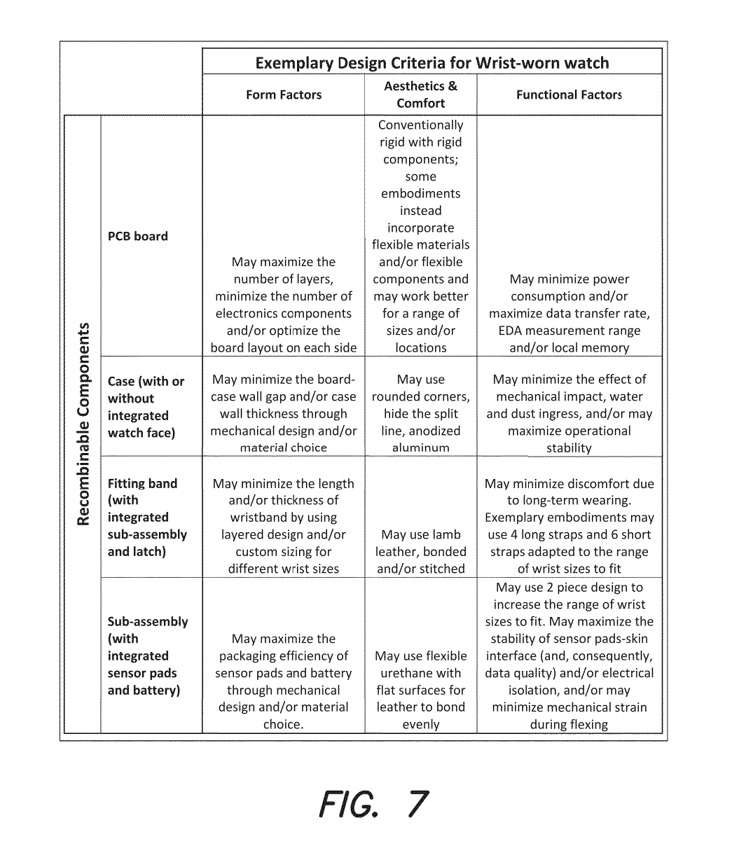

Some embodiments may provide wearable engineered designs that may adapt technologies into recombined shapes and sizes appropriate for materials, technologies, fashions of the time for various demographic and/or anatomical differences. Women, for instance, are smaller on average than men, and have different fashion sensibilities and anatomies. By recombining different components, some embodiments may be engineered to balance the physiology and comfort of the wearer. Since the physiology can exhibit large differences in measurements, technologies may be made to fit the individual wearer for superior signal quality in some embodiments. In some embodiments of designed electronics, data quality and comfort of the wearer may be adjusted with engineering and/or manufacturing constraints. FIG. 7 illustrates exemplary design criteria that may influence the design and/or configuration of components in some embodiments.

In some embodiments incorporating wearable biosensors, physiological data may be collected continuously from the body. Some embodiments may account for specific anatomical constraints in the arrangement of device components within a wearable article. For instance, the top and bottom of the wrist show anatomical differences that reflect the location of the radial and ulnar arteries and the associated vasculature. In some embodiments, a wrist-based design may account for the data quality differences between locations and how the necessary components for the function of the device may be rearranged and relocated. The implementations described herein represent illustrative examples only. Similar configurations designed to balance physiology and anatomy for wearable comfort may be created in some embodiments for the wearer's head, arm, torso, leg, foot, hand, fingers, toes, face, neck, stomach, lungs, throat, intestines, and/or sexual organs, among others.

In some embodiments, unique external shapes may be imposed while accommodating various configurations of components in a wearable article. Curves and lines seen from the outside may enhance the discreteness of the electronic components present inside the article. For instance, in some embodiments, a tear drop or hourglass outline may be imposed rather than a straight watchband, involving an arrangement of device components that is also fit to the particular bodily organs, glands, vasculature, etc., being measured.

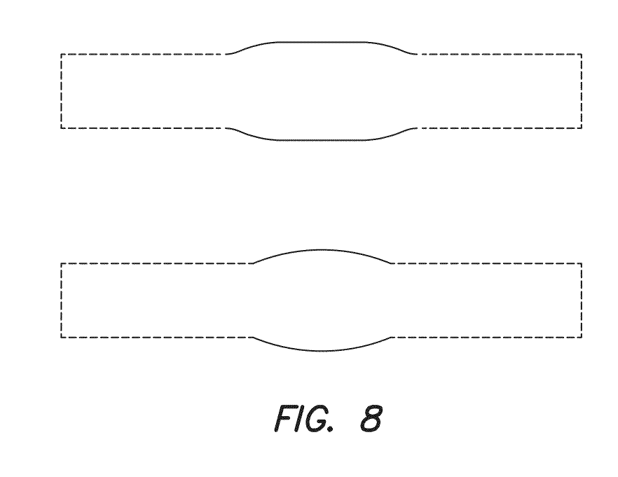

In some embodiments, for example, a watch band housing one or more biometric sensors and/or other electronic components may have one or more sections housing the electronic components that are wider and/or thicker than other sections of the band. For example, in some embodiments, some or all of the electronic components housed in the watch band may be placed at a location in the watch band to be worn against the fleshy underside of the wrist, and this portion of the band may be widened compared with narrower portions of the band worn on the sides of the wrist. In some embodiments, the bulkier portions of the band housing the electronic components may thus be discretely obscured from view by the typical positioning of the wearer's wrist, while the narrower portions of the band may be more visible at the sides and/or top of the wrist. In some embodiments, the widened and/or thickened portion(s) of the band may have sloping and/or rounded contours for aesthetic appeal, as illustrated, for example, in FIG. 8.

In some embodiments, the size of the widened and/or thickened portion(s) may be customized to the size of the wearer's wrist, which in some embodiments may involve customizing the selection and/or arrangement of the electronic components housed in the band. In some embodiments, one or more components such as the battery may be formed in a particular shape and/or size to fit a desirable contour of the wearable article for purposes of aesthetics and/or compatibility with the wearer's anatomy, and/or may be split into multiple smaller components to provide a better fit to the wearer's dimensions and/or to the space and/or form constraints of the wearable article.

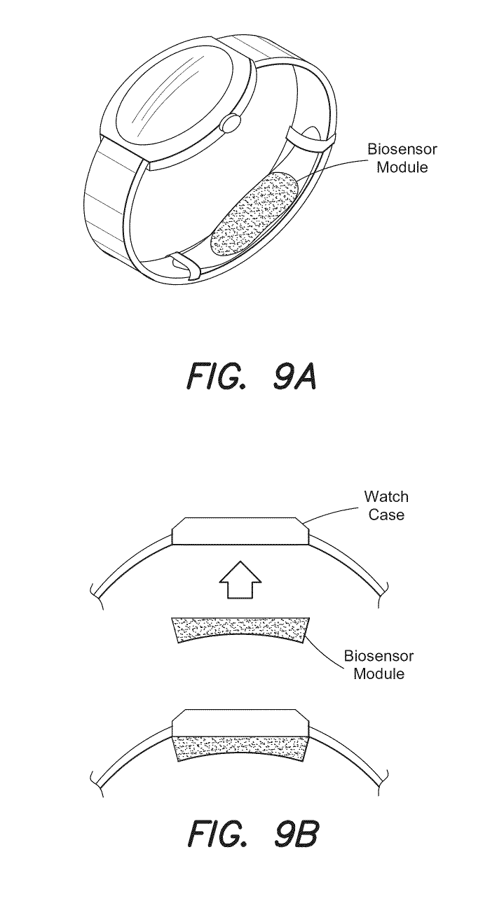

Some embodiments relate to a self-contained biosensor integration module that may include battery, data collection/signal processing electronics, sensing, wireless and/or memory sub-modules. The sub-modules may be packaged into a material that is biocompatible, flexible (to adapt to changing wrist conditions) and strong (to withstand daily wear and tear). The package design may enable breathability during long term wear for comfort, water and dust proofing (for non-standard use cases) and sufficient thermal capacity (to avoid sharp changes in temperature that cause discomfort to user). The package may be designed to have mechanical interfaces that could integrate with many commercial watches. For instance, in one exemplary implementation the module may fit the wristband (at the bottom of the wrist between skin and top of the wristband), as illustrated in FIG. 9A; in another exemplary implementation the module may attach to the watch case (at the top of the wrist between the bottom of the watch case and the skin), as illustrated in FIG. 9B; in another exemplary implementation the module may replace the watch band, and may attach to each side of the watch face, as illustrated in FIG. 9C. In other embodiments, the module may not attach to a watch, but to any other suitable wrist-worn accessory, such as a bracelet. Some embodiments of the biosensor module design may enable a uniform contact with the skin to ensure high data quality even as the entire watch is subjected to daily wear and motion.

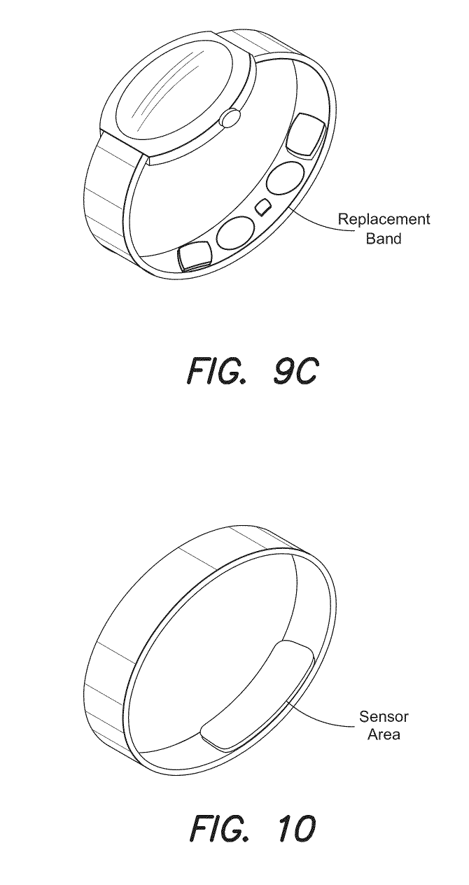

Some embodiments relate to a bioband that may use lightweight, biocompatible and flexible material that conforms to the wrist surface/shape as it expands and contracts to adapt to changing ambient conditions of temperature and humidity and the internal physiological response. An exemplary bioband embodiment is illustrated in FIG. 10. In some embodiments, data collection and signal processing electronics may be constructed out of the rigid-flex PCB board mounted with SMT components that have selected aspect ratios to enable optimal layout of the board to comfortably fit a range of wrist sizes. In some embodiments, the flexible packaging may use one or more curved batteries that may be more compliant to the shape of the wrist. The resulting adaptability combined with the breathable design of the bioband may offer improved comfort and data quality to the user in some embodiments. Comfort may be promoted by ensuring the optimal fit between the band and the wrist surface. Data quality may be promoted by keeping the sensor pad to skin interface independent of any motion experienced during daily wear while not unnecessarily tightening the band elsewhere (and causing discomfort to user). In some bioband embodiments, the sensing module may incorporate silver chloride ink that may be ink-jet printed on the surface of the band (in any custom pattern for optimal data quality and aesthetics). In some embodiments, the sensing module may be located on the bottom of the wrist, where any rigid components may feel more comfortable pressed against fleshy structures as opposed to the bony structures at the sides and/or top of the wrist. In some embodiments, the battery may be the hardest component, and thus may be centered at the bottom of the wrist. On the other hand, more flexible components (including flexible electronics, such as flex-circuits and flexible printed circuit board (PCB) substrates, in some embodiments) may be located at bony parts of the wrist (e.g., the sides and/or top of the wrist) without causing discomfort. Such placement constraints may similarly apply in other embodiments, such as the watch and/or integration module embodiments described above. In some embodiments, sensors may be positioned to be aligned with the location of radial and ulnar arteries to ensure high signal to noise ratio. The bioband may be offered in any color and/or printed pattern for high level of personalization. In some embodiments, a bioband may be used as the base band for a wrist watch, e.g., by attaching a watch face to the bioband.

Some embodiments relate to a biopatch that may be made out of a stretchable and/or flexible substrate with stretchable electronics and battery. Such a biopatch may include some or all of the components described herein as possibly being located in a bioband. In some embodiments, the patch may be applied to the bottom of the wrist at the location of ulnar and radial arteries to maximize signal to noise ratio. However, other embodiments of the biopatch may be configured for placement at any other suitable anatomical location to make suitable measurements from anatomical structures there. In some embodiments, the patch may be adapted to blend into individual skin color or can mimic a decorative/stylistic feature like a tattoo. In some embodiments, the biopatch may be water proof such that it can easily fit into daily wear. In some embodiments, the patch may have a fixed lifetime after which it may shed along with a skin layer or be biodegraded into the skin. In some embodiments, because the patch conforms to the skin during all conditions, the sensing module may maintain a high level of contact with the skin for high data quality. In some embodiments, because the patch is biocompatible and very thin, it may also offer a high degree of comfort.

Some embodiments relate to a bioimplant that may be made out of strong and biodegradable material and in a shape that may require minimal invasive procedure to be inserted into the skin at the bottom of the wrist (at the location of radial and ulnar arteries), or in any other suitable location. In some embodiments, the implant may use the body's heat and motion as a power source and may not require an external power source. In some embodiments, the electronics may be stretchable and optimally placed (e.g., near eccrine sweat glands (for measurement of the innervation of the sweat glands by the adrenal system) and radial and ulnar arteries) to obtain the highest signal to noise ratio. In some embodiments, the implant may have a fixed but reasonable lifetime after which it may be biodegraded and absorbed into the body and released along with the body's waste.

It should be appreciated from the foregoing that some embodiments are directed to a wearable article that houses one or more electronic components such as sensors, and is configured differently for wearers of different physical dimensions, to align one or more of the electronic components with one or more corresponding anatomical structures in the wearer. Some embodiments relate to a band housing one or more electronic components that maintains one or more of the electronic components in contact with the underside of the wearer's wrist.

Some embodiments relate to methods of fitting one or more electronic components such as biometric sensors to a portion of a wearer's anatomy, by taking physical measurements of the wearer, and configuring a wearable article to house the components in a configuration that suitably aligns the components with the wearer's anatomical structures based on the wearer's physical measurements. For example, one type of embodiment is directed to a method for providing a wearable article comprising a flexible band having at least one connecting mechanism configured to connect at least one end of the flexible band to a housing of a watch face, the method comprising: obtaining at least one size measurement of a wrist of a wearer; and selecting a flexible band, based on the at least one size measurement, and positioning one or more biosensors in the flexible band such that, when the watch face housing is worn on an upperside of the wearer's wrist and connected to the flexible band, a first biosensor of the one or more biosensors is positioned against an underside of the wearer's wrist.

The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed is:

1. A wearable article comprising:
a flexible band comprising one or more band segments;
one or more biosensors located in the flexible band;
one or more processing units located in the flexible band; and
at least one connecting mechanism configured to connect at least one end of the flexible band to a housing of a watch face.

2. The wearable article of claim 1, wherein the one or more biosensors are selected from the group consisting of: heart rate, heart rate variability, pulse rate, pulse rate variability, electrocardiography, respiration rate, skin temperature, core body temperature, heat flow, electrodermal, electromyography, electroencephalography, blood pressure, hydration level, muscle pressure, optical reflectance of blood vessels, and oxygen saturation sensors.

3. The wearable article of claim 1, further comprising at least one storage medium, located in the flexible band, storing processor-readable instructions that, when executed by at least one of the one or more processing units, perform a method comprising:
receiving sensor data from the one or more biosensors; and
identifying a physiological state of a wearer of the wearable article by analyzing the received sensor data.

4. The wearable article of claim 3, wherein the method further comprises updating a personalization profile, the personalization profile comprising information relating to:
sensor data indicative of a plurality of physiological states of the wearer; and/or
stimulus that alters a physiological state of the wearer.

5. The wearable article of claim 3, further comprising a wireless transmitter located in the flexible band, wherein the method further comprises transmitting data indicating the identified physiological state of the wearer via the wireless transmitter.

6. The wearable article of claim 3, further comprising a vibration-generating device located in the flexible band, wherein the method further comprises, in response to identifying the physiological state of the wearer, activating the vibration-generating device to alert the wearer to the identified physiological state.

7. The wearable article of claim 6, wherein identifying the physiological state of the wearer comprises determining a level of stress exhibited by the wearer, wherein the vibration-generating device is activated in response to the determined level of stress exceeding a threshold.

8. The wearable article of claim 3, further comprising at least one interface between the one or more processing units and a display of the watch face, wherein the method further comprises, in response to identifying the physiological state of the wearer, displaying an alert on the display of the watch face.

9. The wearable article of claim 3, further comprising a battery located in the flexible band.

10. The wearable article of claim 9, further comprising one or more electrical connections carrying power from the battery to the one or more processing units and to the housing of the watch face.

11. The wearable article of claim 1, wherein a first biosensor of the one or more biosensors is located at a position in the flexible band such that, when the watch face housing is worn on an upperside of a wearer's wrist and connected to the flexible band, the first biosensor is positioned against an underside of the wearer's wrist.

12. The wearable article of claim 11, wherein the first biosensor is located at a position in the flexible band that contacts the wearer's wrist proximate the wearer's radial and/or ulnar artery.

13. The wearable article of claim 11, wherein the flexible band comprises a clasp separating the flexible band into a plurality of band segments, the clasp being located at a position in the flexible band such that, when the watch face housing is worn on the upperside of the wearer's wrist and connected to the flexible band, the clasp is offset from the underside of the wearer's wrist.

14. The wearable article of claim 1, wherein the flexible band comprises a leather band.

15. The wearable article of claim 1, wherein:
the flexible band comprises rubber;
the band further comprises an electrical interconnect between a biosensor of the one or more biosensors and a processor of the one or more processing units; and
the rubber comprising the band is molded around at least the processor and the electrical interconnect.

16. A method of monitoring a physiological state of a wearer via a flexible band connected to a watch face housing worn at the wearer's wrist, the method comprising:
receiving, at one or more processing units located in the flexible band, sensor data from one or more biosensors located in the flexible band; and
analyzing the received sensor data, via the one or more processing units, to compute a score representative of a physiological state of the wearer.

17. The method of claim 16, further comprising:
transmitting the score to a portable electronic device.

18. A watch band comprising:
a band of stretchable material;
a first electronic component and a second electronic component disposed within the band, the first electronic component comprising a biosensor; and
a stretchable electronic interconnect between the first electronic component and the second electronic component.
